(19) United States
(12) Patent Application Publication
(10) Pub. No.: US 2025/0268954 A1
(43) Pub. Date: Aug. 28, 2025

(54) Treatment and/or Prevention of Digestive Disorder by a Bacterial Composition of Propionibacterium Freudenreichii and Bifidobacterium Longum

(71) Applicants: ETH Zurich, Zürich (CH); UNIVERSITÄT ZÜRICH, Zürich (CH)

(72) Inventors: Christophe LACROIX, Kilchberg (CH); Vanesa ROCHA MARTIN, Zürich (CH); Christian BRAEGGER, Zürich (CH)

(73) Assignees: ETH Zurich, Zürich (CH); UNIVERSITÄT ZÜRICH, Zürich (CH)

(21) Appl. No.: 18/580,742

(22) PCT Filed: Jul. 20, 2022
(86) PCT No.: PCT/EP2022/070436

§ 371 Date: Jan. 19, 2024

(30) Foreign Application Priority Data
Jul. 20, 2021 (EP) 21186623.1

(51) Int. Cl. A61K 35/741; A61K 35/745; C12N 1/20; C12R 1/01

(52) U.S. Cl. CPC A61K 35/741; A61K 35/745; C12N 1/205; C12R 2001/01

(57) ABSTRACT

The novel invention is based, at least in part. on the surprising finding that the combination of bacterial strains comprising P. freudenreichii JS27 and B. longum subsp. infantis, particularly B. longum subsp. infantis TPY12-1 can be used in medicine and/or as a food supplement when grown simultaneously. As such and as shown in the description and appended examples, growth and viability of bacterial strains possessing synergistic behavior can be enhanced in the human gut and thus can increase bioavailability as compared with alternative combinations of bacteria.

### CROSS REFERENCE TO RELATED APPLICATION

This application is a national stage application in the U.S. filed under 35 U.S.C. § 371 of pending international application no. PCT/EP2022/069972 filed 17 Jul. 2022, which claims priority to European Patent Application Serial No. EP 21186274.3, filed 17 July Jun. 2021, the entireties of which applications are hereby incorporated by reference herein.

### SEQUENCE LISTING

The instant application contains a Sequence Listing which has been submitted electronically in XML format and is hereby incorporate by reference in its entirety. Said XML copy, created on Jul. 19, 2024, is named 8445-0019_Sequence_Listing.xml and is 2602000 bytes in size.

### FIELD OF THE INVENTION

The present invention relates to a bacterial composition and a method for co-cultivating such a composition. The invention further relates to the use of the composition for the treatment and/or prevention of a digestive disorder. Use of the composition as a food or food-ingredient is also provided.

### BACKGROUND OF THE INVENTION

Infant Colic (IC), a functional gastrointestinal disorder, has been suggested to be caused by an imbalanced composition of the gut microbiota with high abundance of enterobacteria and clostridia which could produce excessive intra-gastrointestinal gas and symptoms of bloating and intestinal inflammation (Savino et al. 2017; Gupta 2002; Lehtonen et al. 1994; Zeevenhooven et al. 2017 and 2018). Moreover, colicky infants experience prolonged and inconsolable crying, which are frequent trigger for abusive head trauma (“Shaken Baby Syndrome”) (Talvik et al. 2008; Barr et al. 2006). Identification of the gut microbiota in different sample groups, healthy control and breast-fed or formula-fed colic infants, confirmed higher abundance of gas-producing bacteria (e.g. saccharolytic Clostridium and Enterobacteriaceae) in the second group (de Weerth et al. 2013; Savino et al. 2009; Lehtonen et al. 1994). Those bacteria produce hydrogen (H₂) and the gas accumulation could contribute to bloating and stomach cramps (Fischbach and Sonnenburg 2011; R. Macfarlace and Gibson 1997; Mckay et al. 1982; Suzuki et al. 2018). Other symptoms suggesting a gastrointestinal disorder through gas accumulation in colicky infants include abdominal distention, flatulence and flexed legs (Gupta 2002; Hyman et al. 2006).

Moreover, according to Pham et al. 2017, higher abundance of H₂-producing lactate-utilizing bacteria Anaerobutyricum hallii and/or Veillonella have been observed in colic infants in comparison to healthy infants. H₂ accumulation, and thus bloating and crying of infants was suggested to be caused by an imbalance in H₂ production from lactate.

Probiotics are living microorganism which when administered in adequate amounts confer a health benefit on the host. Probiotics can be beneficial if consumed in sufficient quantities, and can thus be used against pathogenic bacteria (FAO/WHO 2002). A few digestive disorders affecting the gastrointestinal microbiota of infants are as followed: infant colic, necrotizing enterocolitis and sepsis. Well known probiotics are lactic acid producing strains of bacterial groups of lactobacilli and bifidobacteria. A common problem is the delivery of live bacteria to the gut through the gastrointestinal tract. In currently available bacterial compositions, only a small portion of the composition reaches the gut in a state where bacteria can exhibit their positive effects as food additives and/or on a digestive disease.

Therefore, there is a need for means and methods to improve digestion and the treatment of digesting disorders. In particular, there is a need for novel bacterial compositions showing an improved viability and/or activity for efficacy in the gut after oral intake.

### SUMMARY OF THE INVENTION

The invention relates to a novel bacterial composition comprising viable bacteria of the species Propionibacterium freudenreichii and Bifidobacterium longum subsp. infantis.

In another embodiment, the invention relates to a co-culture of viable bacteria of the species P. freudenreichii and B. longum subsp. infantis.

In a further embodiment, the invention relates to a method of co-cultivating bacteria of the species B. longum subsp. infantis and P. freudenreichii, the method comprising the steps of:
(a) providing a lactose-based cultivation medium;
(b) inoculating the medium of (a) with bacteria of the species P. freudenreichii and B. longum subsp. infantis;
(c) cultivating the inoculated medium of (b).

In a further embodiment, the invention relates to the use of the composition of the invention, the co-culture of the invention and/or the cultivated bacteria of the invention for delivering bacteria of the species P. freudenreichii and B. longum subsp. infantis to the gut after oral intake. A preferred use as provided herein is in the amelioration of digestive conditions in the human gut. It is preferred that the gut is of an infant, preferably of the age of 0 to 3 years.

In a further embodiment, the invention relates to the composition of the invention, the co-culture of the invention and/or the cultivated bacteria of the invention for use in medicine.

In a further embodiment, the invention relates to the composition of the invention, the co-culture of the invention and/or the cultivated bacteria of the invention for use in treating and/or preventing a digestive disorder. The digestive disorder may be lactose intolerance, colic, intestinal discomfort, intestinal pain, visceral sensitivity or intestinal cramp. In some embodiments, digestive disorder described herein is at least one selected from the group consisting of: lactose intolerance, colic, intestinal discomfort, intestinal pain, visceral sensitivity, intestinal cramp and irritable bowel syndrome. It is preferred within the present invention that the composition, co-culture or the cultivated bacteria for use as provided herein is formulated for oral administration. It is furthermore preferred that the composition, co-culture or the cultivated bacteria for use as provided herein is to be administered to an infant, preferably an infant of the age of 0 to 3 years.

It is preferred that the bacteria of the species P. freudenreichii are of the strain P. freudenreichii JS27. It is preferred that the bacteria of the species B. longum subsp. infantis are of the strain B. longum subsp. infantis CECT7210 IM1® (B. infantis IM1®) and/or TPY12-1.

The novel composition provided herein can be used in medicine and/or as a food supplement or food ingredient. As such and as shown in the appended examples, the invention is based, at least in part, on the surprising finding that the combination of bacteria of the species P. freudenreichii and B. longum subsp. infantis leads to a synergistic effect on growth and viability in the infant gut and thus an increased bioavailability as compared with alternative combinations of bacteria.

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

Accordingly, the invention relates to, inter alia, the following embodiments:
1. A bacterial composition comprising viable bacteria of the strain P. freudenreichii JS27 and of the species Bifidobacterium longum subsp. infantis.
2. The composition of embodiment 1, wherein the bacteria of the strain P. freudenreichii JS27 is a bacterium comprising a 16S rDNA sequence as defined by SEQ ID NO: 7 or a sequence having at least 95% sequence identity to SEQ ID NO: 7, wherein the P. freudenreichii JS27 maintains being capable of growing in a colon and wherein post-stress survival is improved by co-culture of the bacterium of the species Bifidobacterium longum subsp. infantis.
3. The composition of embodiment 2, wherein the bacteria of the strain P. freudenreichii JS27 is a bacterium comprising a sequence as defined by SEQ ID NO: 7.
4. The composition of any one of the embodiments 1 to 3, wherein the bacteria of the species B. longum subsp. infantis are of the strain B. longum subsp. infantis TPY12-1.
5. A co-culture of viable bacteria of the strain P. freudenreichii JS27 and of the species Bifidobacterium longum subsp. infantis.
6. The co-culture of embodiment 5, wherein the bacteria of the strain P. freudenreichii JS27 is a bacterium comprising a sequence as defined by SEQ ID NO: 7 or a sequence having at least 95% sequence identity to SEQ ID NO: 7, wherein the P. freudenreichii JS27 maintains being capable of growing in a colon and wherein post-stress survival is improved by co-culture of the bacterium of the species Bifidobacterium longum subsp. infantis.
7. The co-culture of embodiment 6, wherein the bacteria of the strain P. freudenreichii JS27 is a bacterium comprising a sequence as defined by SEQ ID NO: 7.
8. The co-culture of any one of embodiments 5 to 7, wherein the bacteria of the species B. longum subsp. infantis are of the strain B. longum subsp. infantis TPY12-1.
9. A method of co-cultivating bacteria of the strain P. freudenreichii JS27 and of the species B. longum subsp. infantis, the method comprising the steps of:
providing a cultivation medium;
inoculating the medium of (a) with bacteria of the strain P. freudenreichii JS27 and of the species B. longum subsp. infantis, preferably of the strain B. longum subsp. infantis TPY12-1;
cultivating the inoculated medium of (b).
1. Use of the composition of any one of embodiments 1 to 4, or use of the co-culture of any one of embodiments 5 to 9 for delivering bacteria of the strain P. freudenreichii JS27 and of the species B. longum subsp. infantis to the gut, preferably after oral intake.
2. Use of the composition of any one of embodiments 1 to 4, or use of the co-culture of any one of embodiments 5 to 9 in the amelioration of digestive conditions in the human gut.
3. The use of embodiment 10 or 11, wherein the gut is of an infant.
4. The use of embodiment 12, wherein the infant is of the age of 0 to 3 years.
5. The composition of any one of embodiments 1 to 4 or the co-culture of any one of embodiments 5 to 9 for use in medicine.
6. The composition of any one of embodiments 1 to 4 or the co-culture of any one of embodiments 5 to 9 for use in treating and/or preventing a digestive disorder.
7. The composition or co-culture for use of embodiment 15, wherein the digestive disorder is lactose intolerance, colic, intestinal discomfort, intestinal pain, visceral sensitivity or intestinal cramp.
8. The composition or co-culture for use of embodiment 15 or 16, wherein the composition, co-culture or the cultivated bacteria is formulated for gastrointestinal administration.
9. The composition or co-culture for use of embodiment 17, wherein the gastrointestinal administration is oral administration.
10. The composition, co-culture or the cultivated bacteria for use of any one of embodiments 15 to 18, wherein the composition or the co-culture is to be administered to an infant.
11. The composition or the co-culture for use of embodiment 19, wherein the infant is of the age of 0 to 3 years.
12. Use of the composition of any one of embodiments 1 to 4, or the co-culture of any one of embodiments 5 to 9 in a food product.

Accordingly, in one embodiment, the present invention relates to a bacterial composition comprising viable bacteria of the species Propionibacterium freudenreichii and Bifidobacterium longum subsp. infantis. As used herein, the term “viable bacteria”, refers to live bacteria, which are metabolically or physiologically active. Within the present invention, the composition comprises bacteria of the species P. freudenreichii, preferably of the strain P. freudenreichii JS27.

The inventors found that the combination of P. freudenreichii JS27 and Bifidobacterium longum subsp. infantis has specific synergistic effects that go beyond the sum of the two bacteria and beyond what the skilled person would expect from this combination. The inventors found (see FIG. 5) that for a combination of P. freudenreichii JS27 growth was much higher (>two-fold) and metabolite production was increased in culture media containing fermentation effluent (20 and 40%) compared to P. freudenreichii JS DSM 7067. Growth is a key pre-requirement for efficient colonization and high metabolic effect in the gut environment. Furthermore, P. freudenreichii JS27 and Bifidobacterium longum subsp. infantis synergistically improve each other's survival and growth (Example 2 and 3). This synergy is particularly pronounced for P. freudenreichii JS27 since other Propionibacterium freudenreichii such as P. freudenreichii JS DSM 7067 do not have comparable colonization properties in the gut. P. freudenreichii JS DSM 7067 does not substantially grow in experimental settings at 37° C. (see Example 6), therefore this synergistic effect was not to be expected for the person skilled in the art.

Accordingly, the invention is at least in part based on the synergistic effect of P. freudenreichii JS27 and Bifidobacterium longum subsp. infantis on the viability and activity in the gut.

In certain embodiments, the composition of the invention also comprises bacteria of the species B. longum subsp. infantis, preferably of the strain B. longum subsp. infantis CECT7210 IM1® or TPY12-1. As demonstrated in the appended examples, the invention is at least in part based on the surprising finding that a bacterial composition comprising viable bacteria of the species Propionibacterium freudenreichii and Bifidobacterium longum subsp. infantis shows an increased viability as compared to other bacterial compositions. In particular, it was surprisingly shown that the simultaneous cultivation of propionibacteria and bifidobacteria strains promoted growth of B. longum subsp. infantis in in vitro conditions mimicking the infant gut.

This effect is particularly pronounced in combinations comprising B. longum subsp. infantis TPY12-1, since the growth and metabolic activity in culture media containing 20 or 40% fermentation effluent from an infant gut fermentation model is higher (30 to 100% increase) compared to the other B. longum strains (FIG. 6).

As such, the data provided herein plausibly demonstrates that a composition or co-culture comprising these two bacteria leads to an increased availability in the human gut, in particular after gastrointestinal administration. The term “gastrointestinal administration”, as used herein, refers to route of administration in which the administered agent reaches the gastrointestinal tract in a substantial amount. In some embodiments, the gastrointestinal administration described herein is at least one route of administration selected from the group consisting of: oral administration, rectal administration, gastric feeding tube, gastrostomy, duodenal feeding tube and enteral administration. In some embodiments, rectal administration is achieved by a suppository. In some embodiments, oral administration is achieved by a capsule or a tablet.

Further, the invention relates to a co-culture of viable bacteria of the species Propionibacterium freudenreichii and Bifidobacterium longum subsp. infantis. As used herein, the term “co-culture”, refers to a physical embodiment comprising or containing the composition of the invention comprising Propionibacterium freudenreichii and Bifidobacterium longum subsp. infantis, preferably wherein one or both bacterial species are in a growth phase. The co-culture of the invention comprises bacteria of the species P. freudenreichii, preferably of the strain P. freudenreichii JS27. The co-culture of invention comprises bacteria of the species B. longum subsp. infantis, preferably of the strain B. longum subsp. infantis CECT7210 IM1® or TPY12-1.

The present invention further provides a method of co-cultivating bacteria of the species B. longum subsp. infantis and P. freudenreichii, the method comprising the steps of a) providing a lactose-based cultivation medium, b) inoculating the medium of (a) with bacteria of the species P. freudenreichii and B. longum subsp. infantis, and c) cultivating the inoculated medium of (b). As used herein, the term “cultivation medium” refers to a liquid or gel designed to support the growth and/or maintenance of bacteria. Any cultivation medium can be used within the present invention as long as it is able to support the growth of the bacteria of the species B. longum subsp. infantis and P. freudenreichii and preferably lactose based. Cultivation media as used herein can vary, inter alia, in pH, nutrient source, such as glucose concentration, lactate content, protein content, amino acid content, short-chain fatty acid content and/or growth factor content. The term “growth factor” refers to supplements which enhance the growth of bacteria in the cultivation medium. As used herein, the term “lactose-based cultivation medium” refers to a cultivation medium comprising bacteria, in particular of the species B. longum subsp. infantis and P. freudenreichii, which comprises lactose as main energy source. However, the medium may comprise one or more further energy sources such as glucose or fructose.

As such, the medium for culturing is not particularly limited, and a medium usually used for culture of such bacteria can be appropriately modified as required, and used, preferably the main energy and carbon source is lactose. However, as a primary or additional carbon source, for example, saccharides such as galactose, glucose, fructose, mannose, cellobiose, maltose, sucrose, trehalose, prebiotic oligosaccharides, such as fructooligosccharides, galactooligosaccharides and human milk oligosaccharides, starch, starch hydrolysate, and blackstrap molasses can be used according to the present invention. As a nitrogen source, organic nitrogen sources may be used, for example yeast extract, amino acids, peptones, protein hydrolysates. Further, as inorganic salts, for example, sodium chloride, potassium chloride, potassium phosphate, magnesium sulfate, calcium chloride, calcium nitrate, manganese chloride, ferrous sulfate, and so forth can be used. Furthermore, organic components such as peptone, soybean flour, defatted soybean meal, meat extract, and yeast extract may also be used.

Prior to inoculating the lactose-based medium with the bacteria, the bacteria may each individually be grown to provide a suitable starting population of bacteria. The skilled person is aware of means and methods suitable for individual growth of bacteria of the species P. freudenreichii and B. longum subsp. infantis. Exemplary media suitable for such use within the present invention may be based on yeast extract sodium lactate medium (YEL), Wilkens-Chalgren agar, or Man-Rogosa-Sharpe (MRS) broth.

The method of the invention further comprises the step of inoculating the medium with bacteria of the species P. freudenreichii and B. longum subsp. infantis. The term “inoculating” refers to the transfer of at least one bacterial cell able to proliferate from a stock or pre-culture to the lactose-based cultivation medium.

Further, the method of the invention comprises the step of cultivating the inoculated medium. The term “cultivating” refers to the maintenance and/or proliferation of the bacteria in the medium. Cultivation may be carried out for any duration allowing for proliferation of the bacteria. For example, the medium comprising the bacteria may be cultivated for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86,87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or up to 130 h. It is preferred that incubation lasts for 24 to 120 h, more preferably for 48 to 96 h, even more preferably for 72 h. Incubation may be carried out at any temperature suitable to support the growth of the incubated bacteria. It is preferred, however, that incubation is carried out at a temperature of between 30 and 40° C., preferably at 37° C.

The invention also relates to the use of the composition of the invention, the co-culture of the invention or the cultivated bacteria of the invention for delivering bacteria of the species B. longum subsp. infantis and P. freudenreichii to the gut after oral intake. The composition of the invention, the co-culture of the invention or the cultivated bacteria of the invention are also provided as part of a suppository, in particular an infant suppository. The use of the suppository is also provided herein. As shown in the appended examples, bacteria of the species B. longum subsp. infantis and P. freudenreichii show an increased survival in conditions resembling the human gut after having been co-cultured. This surprising synergistic behavior results in an improved usability of the composition, co-culture and/or the co-cultured bacteria according to the invention over available compositions or co-cultures in that the availability of viable bacteria in the human gut is increased.

In certain embodiments, the invention relates to composition of the invention, the co-culture of the invention or the method of co-cultivating of the invention, wherein the bacteria of the strain P. freudenreichii JS27 is a bacterium comprising a sequence as defined by SEQ ID NO: 7 or a sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% sequence identity to SEQ ID NO: 7, wherein the P. freudenreichii JS27 maintains being capable of growing in a colon and wherein post-stress survival is improved by co-culture of the bacterium of the species Bifidobacterium longum subsp. infantis. The capability of growing in a colon is preferably examined in an assay for colon growth at 37° C., more preferably examined in an assay for infant colon growth at 37° C., more preferably in an assay as described in Example 3. In some embodiments, the post-stress survival described herein describes an assay modelling gastric stress conditions such as an assay as described in Example 3b). The improvement described in the context of post-stress survival, preferably refers to an improvement of at least about 10%, at least about 20%, at least about 30%, at least about 40%, at least about 50%, at least about 60% or at least about 70% of the co-culture compared to the single culture.

In certain embodiments, the invention relates to composition of the invention, the co-culture of the invention or the method of co-cultivating of the invention, wherein the bacteria of the strain B. longum subsp. infantis TPY12-1 is a bacterium comprising a sequence identical to the sequences characterizing B. longum subsp. infantis TPY12-1 in Bunesova V. et al. 2016 (Bunesova, V., Lacroix, C., Schwab, C. 2016. Fucosyllactose and L-fucose utilization of infant Bifidobacterium longum and Bifidobacterium kashiwanohense. BMC Microbiol 16, 248) or a sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% sequence identity to the sequences characterizing B. longum subsp. infantis TPY12-1 in Bunesova V et al. 2016 (Bunesova, V., Lacroix, C., Schwab, C. 2016. Fucosyllactose and L-fucose utilization of infant Bifidobacterium longum and Bifidobacterium kashiwanohense. BMC Microbiol 16, 248), wherein the B. longum subsp. infantis TPY12-1 maintains being capable of improving post-stress survival of the bacterium of the strain P. freudenreichii JS27 by co-culture.

“Percent (%) sequence identity” with respect to a reference sequence is defined as the percentage of residues in a candidate sequence that are identical with the residues in the reference sequence, after aligning the sequences and introducing gaps, if necessary, to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The differences compared to the reference sequence (e.g. reference genome) may be the result of natural or engineered mutations that do not or not substantially limit the technical effect(s) described herein. As such, the mutations can comprise insertions, deletions and/or replacements of nucleotides in the reference genome sequences as defined by SEQ ID NO: 7 and/or the sequences characterizing B. longum subsp. infantis TPY 12-1 in Bunesova V et al. 2016 (Bunesova, V., Lacroix, C., Schwab, C. 2016. Fucosyllactose and L-fucose utilization of infant Bifidobacterium longum and Bifidobacterium kashiwanohense. BMC Microbiol 16, 248).

In a further embodiment, the invention relates to the use of the composition of the invention, the co-culture of the invention or the cultivated bacteria of the invention in the amelioration of digestive conditions in the human gut. Such use may be non-medical but may generally relate to the improvement of the well-being of the subject using the composition of the invention, the co-culture of the invention or the cultivated bacteria of the invention. The term “subject” as used herein can be any animal having a gastrointestinal tract suitable for hosting bacteria such as a microbiome. In preferred embodiments, the subject is a human. Accordingly, the “digestive condition” as used herein may generally relate to a subjective feeling without necessarily being related to a true medical disorder or disease. As such, the non-medical use of the composition of the invention, the co-culture of the invention or the cultivated bacteria of the invention may improve the subjective feeling of a subject to be affected by a digestive condition.

In another embodiment, the invention relates to the composition of the invention, the co-culture of the invention, or the cultivated bacteria of the invention for use in medicine.

In a further embodiment, the invention relates to the composition of the invention, the co-culture of the invention or the cultivated bacteria of the invention for use in treating and/or preventing a digestive disorder.

As used herein, the term “digestive disorder”, refers to a disorder related to the gastrointestinal tract. The digestive disorder may be lactose intolerance, colic, intestinal discomfort, intestinal pain, visceral sensitivity, and/or intestinal cramp.

Within the present invention, the composition of the invention, the co-culture of the invention or the cultivated bacteria of the invention may be administered to an infant. Within the present invention, the infant may be a human subject of the age of 0 to 3 years. The term “treating” (and its grammatical variations thereof such as “treat” or “treating”), as used herein, refers to a clinical intervention in an attempt to alter the natural course of the individual being treated, and can be performed either for prophylaxis or during the course of clinical pathology. The term “preventing” refers to, but is not limited to, inhibition or the averting of symptoms associated with a particular disease or disorder. Desirable effects of treatment include, but are not limited to, alleviation of symptoms, diminishing of any direct or indirect pathological consequences of the disorder, or preventing occurrence or recurrence of at least one of the disorders, such as lactose intolerance, colic, intestinal discomfort, intestinal pain, visceral sensitivity, or intestinal cramp. Preferably, the effects of treatment and/or prevention include alleviation of infant colic. Accordingly, the invention is at least in part based on the finding that a bacterial composition comprising P. freudenreichii and B. longum subsp. infantis can treat and/or prevent digestive disorders such as colics of an infant. The combination of the two bacteria species can prevent lactate accumulation and its conversion to H₂ by lactate-utilizing H₂-producing bacteria, which can be health beneficial by treating and/or preventing bloating, intestinal discomfort, and pain.

In a particular embodiment, the invention relates to the composition, co-culture or the cultivated bacteria, wherein the composition, co-culture or the cultivated bacteria is formulated for oral administration or anal administration. The term “oral administration”, as defined herein, refers to the intake of the composition, co-culture or the cultivated bacteria of the invention through the mouth. In order to ensure a suitable amount of viable bacteria in the gut of the subject, the composition, co-culture or the cultivated bacteria is formulated to allow for survival of a sufficient number of bacteria after passing through the upper gastrointestinal tract, in particular the stomach. In this regard, it was surprisingly found by the inventors that the composition, co-culture or the cultivated bacteria of the invention allows for enhanced survival of the bacteria in conditions resembling the infant gut based on a synergistic effect of the bacterial species of the composition, co-culture or the cultivated bacteria of the invention.

The composition, co-culture or the cultivated bacteria of the invention may be formulated as a liquid solution, suspension or powder for medical or non-medical use. For example, the composition, co-culture or the cultivated bacteria may be formulated as dietary product for medical or non-medical use. The composition, co-culture or the cultivated bacteria may also be formulated as comprising freeze-dried bacteria. One particular form is a suppository for anal administration.

The general methods and techniques described herein may be performed according to conventional methods well known in the art and as described in various general and more specific references that are cited and discussed throughout the present specification unless otherwise indicated. See, e.g., Rinttilä et al. 2004,Herve et al. 2007, Furet et al. 2009, Mozzetti et al. 2012, Doo et al. 2017, Pham et al. 2019, or Rocha Martin et al. 2019.

While aspects of the invention are illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below. The invention also covers all further features shown in the figures individually, although they may not have been described in the previous or following description. Also, single alternatives of the embodiments described in the figures and the description and single alternatives of features thereof can be disclaimed from the subject matter of the other aspect of the invention.

### BRIEF DESCRIPTION OF DRAWINGS

FIG. 1: Hypothetical scheme of the mechanism of H₂ production in Infant Colic (IC). H₂-producing bacteria convert carbohydrates and lactate into H₂ gas leading to bloating and associated pain.

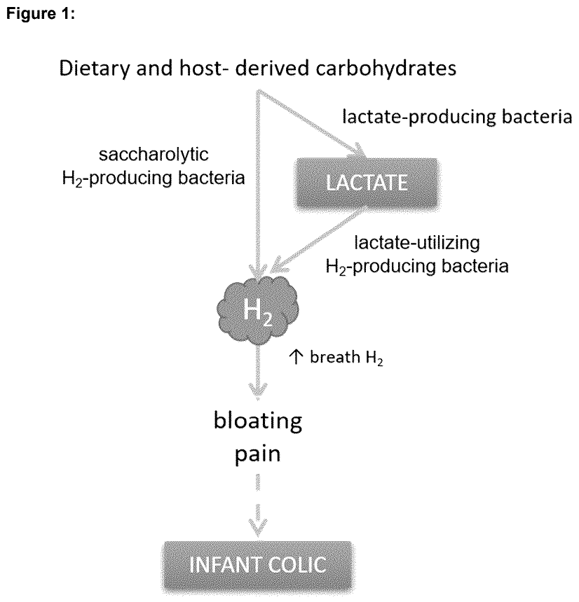

FIG. 1A/B: Growth and metabolism of single-and co-cultures of Bifidobacterium spp. or Lacticaseibacillus rhamnosus LGG® with Propionibacterium freudenreichii in media mimicking infant proximal colon conditions. Strain abundance (A1-E1 and B3-E3) and carbohydrate utilization and metabolite formation (A2-E2 and B4-E4) of single cultures (FIGS. 1-2) of P. freudenreichii JS27 (A), B. longum subsp. infantis TPY 12-1 (B), Bifidobacterium longum subsp. infantis CECT7210 IM1® (C), Bifidobacterium animalis subsp. lactis BB-12® (D) and Lacticaseibacillus rhamnosus LGG® (E) and respective co-cultures with P. freudenreichii JS27 (figures B to E 3-4) during incubation for 3 days at 37° C. in fermentation media supplemented with 60% (v/v) of filter sterilized fermentation effluent collected from an in vitro continuous PolyFermS fermentation model mimicking the proximal colon of a two months old bottle-fed healthy baby. Mean values and standard deviations from experiments done in triplicates are shown.

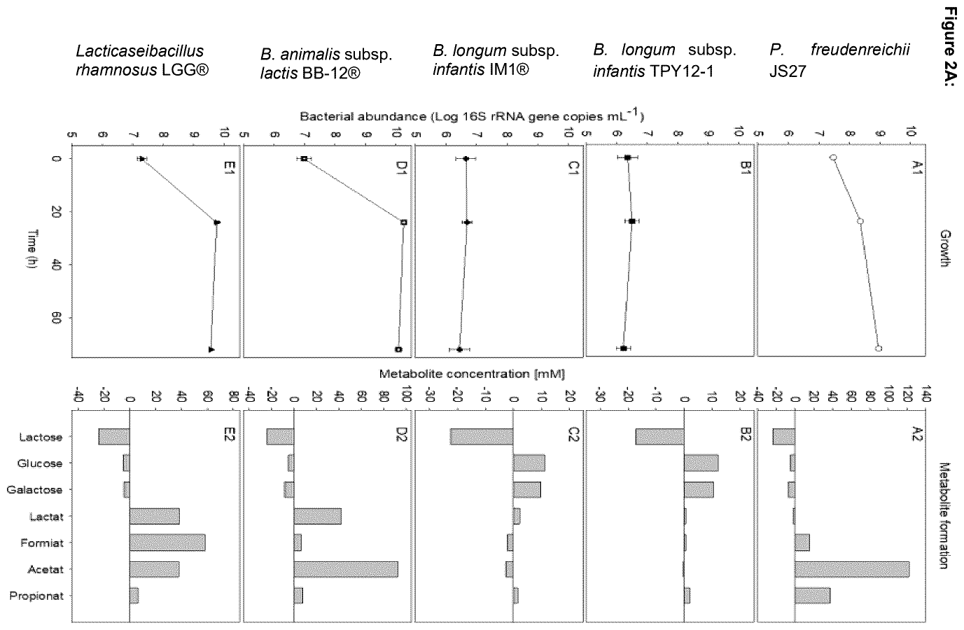

FIG. 2: Preference of metabolism of lactate over lactose by P. freudenreichii strains isolated from dairy foods possessing β-galactosidase activity. Mean carbon utilization and metabolite formation by P. freudenreichii strains (difference between duplicates were <3.5 mM for all tested strains) after 24 (black) and 48 h (grey) of incubation in YEL media containing 70 mM of DL-lactate and 25 mM of lactose as carbon sources.

FIG. 4: Survival to gastric conditions of single cultures of Bifidobacterium spp. and Lacticaseibacillus rhamnosus LGG®, and single and co-cultures of P. freudenreichii JS27. Mean bacterial counts after incubation for 72 h in lactose-based media (T₀: pre-stress bacterial counts) and after 15 min exposure to gastric conditions (T₁: post-stress survival). ns: p>0.05; ***: p<0.001; ****: p<0.0001.

FIG. 5: Growth at 24 and 48 h, lactate utilisation and propionate and acetate formation of P. freudenreichii strains JS27 and JS DSM 7067 in YEL broth containing 55 mM sodium-DL-lactate, and 20% (YEL 80%) or 40% (YEL 60%) v/v of fermentation effluent.

FIG. 6: Growth at 24 h and glucose utilisation and acetate, lactate and formate formation of B. longum subsp. infantis TPY 12-1 (A), B. longum subsp. infantis CECT7210 IM1® (B), B. longum subsp. longum 35624® (C), and B. longum W11® (D) in mWCSP culture media supplemented with prebiotics (GOS+FOS) and fermentation effluent. Water was replaced in mWCSP media by fermenter effluent in 20% (mWCSP 80%+GOS+FOS) and 40% v/v (mWCSP 60%+GOS+FOS).

Furthermore, in the claims the word “comprising” does not exclude other elements or steps, and the indefinite article “a” or “an” does not exclude a plurality. A single unit may fulfill the functions of several features recited in the claims. Any reference signs in the claims should not be construed as limiting the scope. As used herein, “and/or” should be understood to mean either one, or both alternatives.

The following are examples of methods and compositions of the invention. It is understood that various other embodiments may be practiced, given the general description provided above.

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those skilled in the art that the techniques disclosed in the examples which follow represent techniques used in the present invention to function well in the practice of the invention, and thus can be considered to constitute preferred modes for its practice. However, those skilled in the art should appreciate, in light of the present disclosure that many changes can be made in the specific embodiments which are disclosed and still obtain a like or similar result without departing from the spirit and scope of the invention. Several documents including patent applications, manufacturer's manuals and scientific publications are cited herein. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### EXAMPLES

### Example 1

The bacteria were grown in medium.

The bacterial strains were obtained from the strain collection of the Laboratory of Food Biotechnology (LFB; ETH-Zurich) and/or isolated from commercial products as indicated in the following table 1.

TABLE 1
Strains

| | | Isolation Source and |
|---|---|---|
| Strain name | Origin | Comments |
| Propionibacterium | cheese | LFB strain ID: BT-1205. |
| freudenreichii JS27 (as | | |
| characterized in MIESCHER, | | |
| Susanne. Antimicrobial and | | |
| autolytic systems of dairy | | |
| propionibacteria. 1999. Diss. | | |
| ETH No. 13486) (comprising | | |
| SEQ ID NO: 7) | | |
| Propionibacterium | cheese | LFB strain ID: BT-2764. |
| freudenreichii JS DSM 7067 | | Ex-HOLDBAC YM-B |
| | | (commercial protective |
| | | culture). |
| Bifidobacterium longum | Isolated from | Commercial product: |
| subsp. longum 35624 ® | resected | Alflorex ® capsules. |
| | human | Precisionbiotics, Cork, |
| | healthy | Ireland. |
| | gastrointestinal | |
| | tissue. | |
| Bifidobacterium longum W11 | intestinal | Commercial product: |
| | microbiota of | Proteflor ® IBS. |
| | a healthy | Sundyota Numandis |
| | man | Probioceuticals Pvt. Ltd. |
| | | Gujarat, India. |
| Bifidobacterium longum | infant feces, | LFB strain ID: BT-4325. |
| subsp. infantis TPY12-1 | 6 months old | |
| (as characterized in Bunesova, | | |
| V., Lacroix, C., Schwab, C. | | |
| 2016. Fucosyllactose and | | |
| L-fucose utilization of | | |
| infant Bifidobacterium longum | | |
| and Bifidobacterium | | |
| kashiwanohense. | | |
| BMC Microbiol 16, 248) | | |
| Bifidobacterium longum | breast-fed | Commercial product: |
| subsp. infantis CECT7210 | infant feces | Blemil Plus Optimum 3. |
| IM1 ® | | Laboratorios ORDESA |
| | | SL. |
| Bifidobacterium animalis | Chr. Hansen's | Commercial product: |
| subsp. lactis BB-12 ® | collection of | OptiBac Bifidobacteria & |
| | dairy cultures | Fibre. |
| Lacticaseibacillus rhamnosus | fecal samples | LFB Strain ID: BT-1001 |
| LGG ® | of a healthy | and commercially |
| | human adult | available |

P. freudenreichii JS27 (comprising SEQ ID NO: 7) was grown in yeast extract sodium lactate medium (YEL) consisting of:
1% (w/v) trypticase soy broth without dextrose (Becton Dickinson AG, Allschwil, Switzerland);
1% (w/v) yeast extract (Merck, Darmstadt, Germany);
117 mM sodium DL-lactate 50% (Sigma-Aldrich, Buchs, Switzerland).

Bifidobacterium spp. were grown on modified Wilkens-Chalgren medium (mWCSP) (Oxoid, Thermo Fisher Diagnostics AG, Pratteln, Switzerland) supplemented with:
0.5% (w/v) soy peptone (Biolife, Italy);
0.1% (v/v) Tween 80 (Sigma-Aldrich);
0.05% (w/v) L-cysteine (Sigma-Aldrich).

Lacticaseibacillus spp. was grown in Man-Rogosa-Sharpe (MRS) broth (BioLife, Switzerland).

Glycerol stocks stored at −80° C. were re-activated on agar plates and incubated in anaerobic jars (Mitsubishi AnaeroPack, Thermo Fisher Diagnostics AG, Pratteln, Switzerland) containing the AnaeroGen system (Oxoid, Thermo Fisher Diagnostics AG). Bifidobacterium and Lacticaseibacillus were incubated at 37° C. for two (2) days and Propionibacterium for five (5) days.

Subsequently, a single colony was picked, transferred into conical polypropylene tubes containing 10 mL of sterile broth and Bifidobacterium and Lacticaseibacillus were incubated for 48 h and Propionibacterium for 72 h at 37° C. Strains were sub-cultured twice in liquid media before being used as working cultures.

### Quantification of Bacterial Abundance

DNA isolation from single-and co-cultures and quantitative PCR analysis (qPCR)

Genomic DNA was extracted from bacterial pellets using the Fast DNA SPIN kit for soil (MP Biomedicals, Illkirch, France) according to manufacturer's instructions. Reactions were performed using LightCycler 480 Real-Time PCR System (Roche Diagnostics, Rotkreuz, Switzerland), 5 μL of SensiFASTSYBR No-ROX 2X mix, and 500 nM primers (Biolab Scientifics Instruments SA, Chatel-St-Denis, Switzerland) in a total reaction volume of 10 μL. Thermal cycling started with an initial denaturation step at 95° C. for 3 min, followed by 40 cycles of a two-step PCR at 95° C. for 5 s and at 60° C. for 60 s. Ct values were obtained using automatic baseline and threshold settings provided by the LightCycler 480 Software, Version 1.5. Individual samples were analyzed in duplicates.

To generate standards, PCR amplicons were cloned into the pGEM-T Easy Vector and heterologously expressed in E. coli according to instructions of the supplier (Promega AG, Dübendorf, Switzerland). Standard curves were prepared from ten-fold dilutions of linearized plasmids harboring the target gene of interest. Melting curve analysis was conducted to confirm specificity.

P. freudenreichii (comprising SEQ ID NO: 7) was quantified using primers as followed (Herve et al. 2007):

q5S Trans Fwd
(5′-ATTCCATCGCCCTGAAGGA-3′; SEQ ID NO. 1);
q5S Trans Rev
(5′-TTGATCTGCGTCTTCTGGCC-3′; SEQ ID NO. 2).

Bifidobacterium was quantified using primers as follows (Rinttilä et al. 2004):

bif_F
(5′-TCGCGTCYGGTGTGAAAG-3′; SEQ ID NO. 3);
bif_R
(5′-CCACATCCAGCRTCCAC-3′; SEQ ID NO. 4).

Lacticaseibacillus was quantified using primers as follows (Furet et al., 2009):

F_Lacto 05
(5′-AGCAGTAGGGAATCTTCCA -3′; SEQ ID NO. 5)
R_Lacto 04
(5′-CGCCACTGGTGTTCYTCCATATA-3′; SEQ ID NO. 6)

The linear detection range was between 3.1 and 9.3 log gene copies for P. freudenreichii, 3.5 and 7.5 log gene copies for bifidobacteria, and 3.9 and 8.9 log gene copies for Lacticaseibacillus, and primer efficiency 98, 104, and 101%, respectively.

### Statistical Analysis

Statistical comparison was performed using two-way ANOVA followed by Holm-Sidak correction and was performed using Graph Pad Prism 8.2 (GraphPad Software, Inc. La Jolla, CA).

### Example 2

Single and co-culture growth and metabolism of Bifidobacterium spp. or Lacticaseibacillus rhamnosus LGG® and P. freudenreichii JS27 in media mimicking infant proximal colon conditions

The investigation of the synergistic behavior of the bacteria was performed in in vitro conditions mimicking the infant gut. Evaluation growth and metabolic cross-feeding was performed of following bacterial strains and co-cultures:
P. freudenreichii JS27 (comprising SEQ ID NO: 7)
B. longum subsp. infantis TPY12-1
B. longum subsp. infantis CECT7210 IM1®
B. animalis subsp. lactis Bb12
L. rhamnosus LGG®
B. longum subsp. infantis TPY12-1 and P. freudenreichii JS27 (comprising SEQ ID NO: 7)
B. longum subsp. infantis CECT7210 IM1® and P. freudenreichii JS27 (comprising SEQ ID NO: 7)
B. animalis subsp. lactis BB-12® and P. freudenreichii JS27 (comprising SEQ ID NO: 7)
L. rhamnosus LGG® and P. freudenreichii JS27 (comprising SEQ ID NO: 7)

The evaluation was done in triplicates in 2.2 mL 96-deep-well plates (Milian SA, Vernier/Geneve, Switzerland) covered with Breathe-Easy sealing membranes (Sigma-Aldrich) and incubated in anaerobic jars (Mitsubishi AnaeroPack, Thermo Fisher Diagnostics AG) containing the AnaeroGen system (Oxoid, Thermo Fisher Diagnostics AG) for 3 days at 37° C.

Each well contained 1.6 mL of fresh medium previously designed to mimic the chyme entering the colon of 6-month-old infants (Doo et al. 2017; Pham et al. 2019; Rocha Martin et al. 2019) and containing 60% (v/v) of filter sterilized fermentation effluent. Fermentation effluent was collected from the control reactor of an in vitro continuous fermentation model mimicking the proximal colon of a two months old infant and inoculated with immobilized fecal microbiota from a two months old bottle-fed healthy baby, corresponding to Fermentation 2 described in Pham et al. (2019).

Concentrations of carbohydrates, SCFA and fermentation metabolites in effluent and supplemented fermentation media with 60% effluent are shown in following Table:

TABLE 2
Carbohydrates, SCFA and fermentation metabolites

| | | | Concentrations | [mM] | | | | | |
|---|---|---|---|---|---|---|---|---|---|
| Sample ID | lactose | glucose | galactose | succinate | lactate | formate | acetate | propionate | butyrate |
| media with | 24.3 | 5.1 | 9.2 | 3.9 | 4.2 | 24.1 | 44.2 | 6.5 | 3.6 |
| 60% effluent | | | | | | | | | |
| Fermentation | BDL | BDL | BDL | 1.2 | 1.9 | 18.3 | 60.8 | 7.4 | 7.2 |
| 2 effluent | | | | | | | | | |
| (Pham et al., | | | | | | | | | |
| 2019) | | | | | | | | | |

BDL: below detection limit (detection limit <5 mM)
isobutyrate, isovalerate and valerate <5 mM (not shown)

For single culture experiments, wells were inoculated with 16 μL (1% v/v) of each working culture or each tested culture. For both experiments, 400 μL of samples were collected before incubation and after 24 and 72 h of incubation. Subsequently, samples were centrifuged, and supernatants and cell masses were stored at −20° C. for future analysis.

Preference of metabolism of lactate or lactose by P. freudenreichii strains isolated from dairy foods possessing β-galactosidase activity

P. freudenreichii strains were incubated at 37° C. for 48 h in conical shaped polypropylene tubes containing 900 μL of YEL broth, and two carbon sources 70 mM of DL-lactate and 25 mM of lactose.

From the above P. freudenreichii working culture, 100 μL (10% v/v on final volume) were used for inoculation. Subsequently, samples of 400 μL were taken after 24 and 48 h for the determination of the concentrations of lactate, sugars, SCFA, and intermediate metabolites in the supernatants by high-pressure liquid chromatography analysis with refractive index detection (HPLC-RI; Experiment 2b).

Lactate and carbohydrate utilization and metabolite formation were assessed in duplicates. Difference between duplicates was <3.5 mM for all tested strains.

### High-Pressure Liquid Chromatography Analysis with Refractive Index Detection (HPLC-RI)

Concentrations of lactose, glucose, galactose, acetate, propionate, butyrate, formate, lactate, succinate, isobutyrate, isovalerate, and valerate were determined in the supernatant.

For the analysis, 400 μL of the supernatants were filtered through a 0.45 μm membrane (Millipore AG, Zug, Switzerland), transferred into glass HPLC vials (Infochroma, Hitachi LaChrome, Merck, Dietikon, Switzerland), and sealed with crimp-caps.

The HPLC (Hitachi LaChrome) system was equipped with a refractive index (RI) detector and the used column consisted of two parts (stationary phase):
Security Guard Cartridges Carbo-H column (4×3 mm; Phenomenex Inc., Torrance, CA, USA);
Rezex ROA-Organic Acid H+ column (8%, 300× 7.8 mm; Phenomenex).

The mobile phase consisted of a 10 mM H₂SO₄ (Fluka, Buchs, Switzerland) solvent. The elution was performed at a flow rate of 0.4 mL/min at 25° C. Detection limit was of 5 mM.

### Results

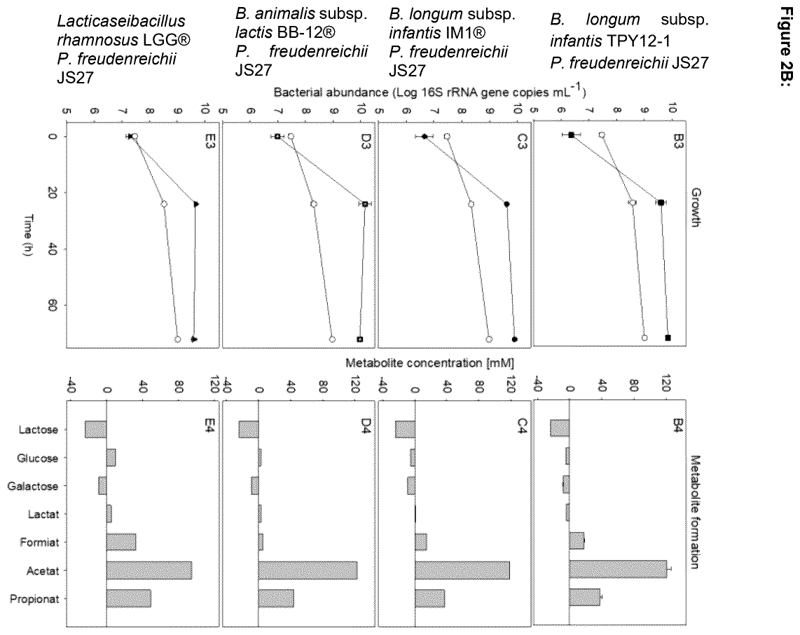

The simultaneous cultivation of propionibacteria and bifidobacteria strains promoted growth of B. longum subsp. infantis in in vitro (fermentation medium) conditions mimicking the infant gut. FIG. 2 A1-2 exhibits the growth of P. freudenreichii JS27 in infant colon conditions and the resulting metabolization of lactate and lactose into propionate, acetate, and CO₂. This would prevent H₂-production by removing substrates and thus feeding both metabolic pathways by which it is produced. P. freudenreichii will compete for the same substrate with lactose-utilizing H₂-producing bacteria (Enterobacteriaceae and Clostridium) and lactate-utilizing H₂-producing bacteria (Veillonella and E. halli) present in the infant colon.

Various propionibacteria strains (JS9, DF28, SM220, JS62, JS27, MS29, JS7, MS32, JS3, SM206, JS23 or JS26) were analyzed upon their metabolite concentration in order to identify the preference of lactose or lactate (FIG. 3). It was observed that lactate is the preferred carbon source over lactose for the P. freudenreichii strains with β-galactosidase activity (FIG. 3). However, to efficiently remove lactose and generate a more efficient competition with fast lactose-degrading bacteria such as Enterobacteriaceae and Clostridium, lactose-consuming bifidobacteria were co-cultured with propionibacteria.

Individually grown B. animalis subsp. lactis BB-12® bacteria converted lactose into acetate, lactate and formate and bacterial abundance was increased with or without the presence of P. freudenreichii JS27 (FIG. 2 C1-4, E1-4).

The drawback of the solely supplementation of B. animalis subsp. lactis BB-12® in milk-fed infants, could be the accumulation and thus high production of lactate. This bacterial property can cause neurotoxicity and cardiac arrhythmia. Moreover, this high abundance of lactate can be consumed by lactate-utilizing H₂-producing bacteria (Veillonella and _{E. hallii}) and produce H₂, which accumulation can lead to bloating, intestinal discomfort, and pain. Lactate accumulation was also observed when Lacticaseibacillus rhamnosus LGG® single culture was grown in infant colon conditions (FIG. 2 F1-2).

B. longum subsp. infantis of the strains TPY12-1 and CECT7210 IM1® were not growing as single cultures (FIG. 2 B1-2, D1-2), whereas co-cultivation with P. freudenreichii JS27 (comprising SEQ ID NO: 7) in infant colon conditions lead to growth of all strains (FIG. 2 B3-4, D3-4). Synergistic behavior of the two co-cultures, wherein Bifidobacterium was cultivated in presence of Propionibacterium, resulted in no accumulation of lactate. Thus, the combination of the two bacteria genera can prevent lactate accumulation and its conversion to H₂ by lactate-utilizing H₂-producing bacteria, which can be health beneficial by treating and/or preventing bloating, intestinal discomfort, and pain.

Strains from B. longum subsp. infantis were the preferred strains in co-culture with P. freudenreichii JS27 (comprising SEQ ID NO: 7), since this composition ensures that the formation of lactate by the Bifidobacterium will occur only in presence of the propionibacteria, which on the other side can utilize lactate produced by bifidobacterial and thus prevent its accumulation. The outcome of these findings is, that the growth of B. longum subsp. infantis strains is promoted and thus show a synergistic behavior in in vitro conditions mimicking the infant gut when P. freundenreichii strain is present while the dependency of bifidobacterial on growth factors produced by propionibacteria enable a balanced growth of the co-culture.

### Example 3

### Introduction

In order to produce a suitable formulation in lactose-based media, a novel combination of bacterial species in presence of propionibacteria was established.

Single and co-culture growth of Bifidobacterium spp. or Lacticaseibacillus rhamnosus LGG® and P. freudenreichii JS27 in lactose-based media

Growth interactions between P. freudenreichii JS27 (comprising SEQ ID NO: 7) and Bifidobacterium or Lacticaseibacillus LGG® in lactose-based media were evaluated in two biological replicates:
P. freudenreichii JS27 (comprising SEQ ID NO: 7) and Bifidobacterium
P. freudenreichii JS27 (comprising SEQ ID NO: 7) and Lacticaseibacillus rhamnosus LGG®.

The biological replicates were grown as pure cultures or in co-culture with P. freudenreichii JS27 (comprising SEQ ID NO: 7) in lactose-based media composed of:
lactose 6.4 g/L (Sigma-Aldrich, Buchs, Switzerland);
whey protein 10 g/L (Emmi, Dagmersellen, Switzerland);
yeast extract 5 g/L (Merck, Darmstadt, Germany);
L-cysteine HCl 0.5 g/L (Sigma-Aldrich);
KH₂PO₄ 3 g/L (Sigma-Aldrich);
NaHCO₃ 9 g/L (Sigma-Aldrich).

Strains were inoculated at 1% v/v (simultaneous inoculation for co-cultures at 1% of each strain) and cultures were incubated for 72 h at 37° C. Viable propionibacteria, bifidobacteria and Lacticaseibacillus were counted after incubation period (T₀: pre-stress bacterial counts) on following agar plates:
Propionibacteria on YEL;
Bifidobacteria on mWCSP;
Lacticaseibacillus on MRS.

Plates were incubated in anaerobic jars (Mitsubishi AnaeroPack, Thermo Fisher Diagnostics AG) containing the AnaeroGen system (Oxoid, Thermo Fisher Diagnostics AG) at 37° C.: bifidobacteria and Lacticaseibacillus for 48 h, and propionibacteria for 5 days.

### Exposure of Single and Co-Cultures to Gastric Stress Conditions

After single or co-culture fermentation in lactose-based media, 20 μL of cell suspensions were exposed in triplicate to gastric stress conditions in 96-well microtiter plates (Bioswisstec AG, Schaffhausen, Switzerland) and incubated 15 min at 37° C. in anaerobic jars (Mitsubishi AnaeroPack, Thermo Fisher Diagnostics AG) containing the AnaeroGen system (Oxoid, Thermo Fisher Diagnostics AG).

Effect of simulated gastric juices was tested by adding 20 μL of cell suspension to 180 μL of filter sterilized 0.1 M HCl solution composed of (Mozzetti et al. 2013):
0.5% (w/v) NaCl;
0.4% (w/v) pepsin (541 U/mg) from porcine gastric mucosa (Sigma-Aldrich) (gastric juice pH 3).

Subsequently, cells were stress exposed for 15 min and the mixtures were 10-fold serially diluted in cPBS. Viable bacteria were counted on agar plates as previously described (T₁: post-stress survival).

### Results

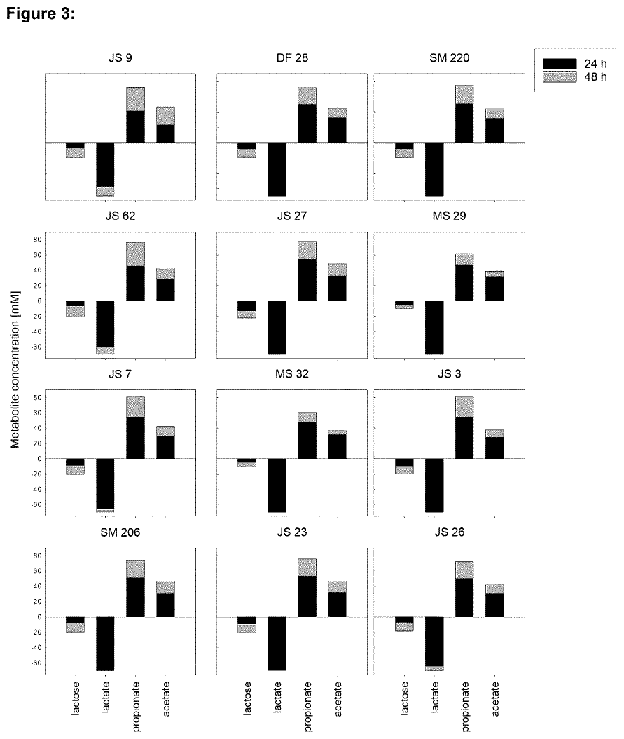

The cells suspensions obtained by pure cultures of P. freudenreichii and in absence of bifidobacteria do not resist gastric stress conditions as described in the method. After exposure to those conditions no viable cells were recovered by plate counting (FIG. 4). The same effect of no cell recovery was observed for the co-cultures of Propionibacterium and B. animalis subsp. lactis BB-12®. Nevertheless, simultaneous cultivation of Propionibacterium and B. longum subsp. infantis TPY12-1 or CECT7210 IM1® and exposure to gastric conditions leads to a higher survival of viable cells of P. freudenreichii JS27 (FIG. 4).

### Example 4

Growth and metabolism of Propionibacterium freudenreichii strains in culture media containing fermentation effluent:

Strains stored in glycerol stocks at −80° C. were reactivated on YEL agar plates and incubated in anaerobic jars containing the AnaeroGen system at 37° C. After 5 days of incubation, a single colony was picked, transferred into conical polypropylene tubes containing 10 mL of sterile YEL broth and incubated for 72 h at 37° C. Strains were cultured twice in liquid media before using as working cultures.

To investigate ability to grow and metabolic activity in presence of filter sterilized fermentation effluent from an in vitro continuous fermentation model mimicking the proximal colon of a two months old infant and inoculated with immobilized fecal microbiota from a two months old bottle-fed healthy baby, corresponding to Fermentation 1 described in Pham et al. (2019), 20 μL of each working culture were used to inoculate wells in 96-well microtiter plates, each well containing 180 μL of YEL broth containing 55 mM sodium-DL-lactate, and different proportions of fermentation effluent. Water was replaced in YEL media by fermenter effluent by 20% (YEL 80%) and 40% (YEL 60%).

Cultures were incubated for 48 h at 37° C. in anaerobic jars containing the AnaeroGen system. Cell growth was assessed in triplicates for each strain by measuring culture optical density at 600 nm (OD600) at 24 and 48 h. Concentrations of substrate, SCFA and intermediate metabolites in pooled supernatant from cultures from same strains and in same media were determined by high-pressure liquid chromatography analysis with refractive index detection (HPLC-RI) after 48 h.

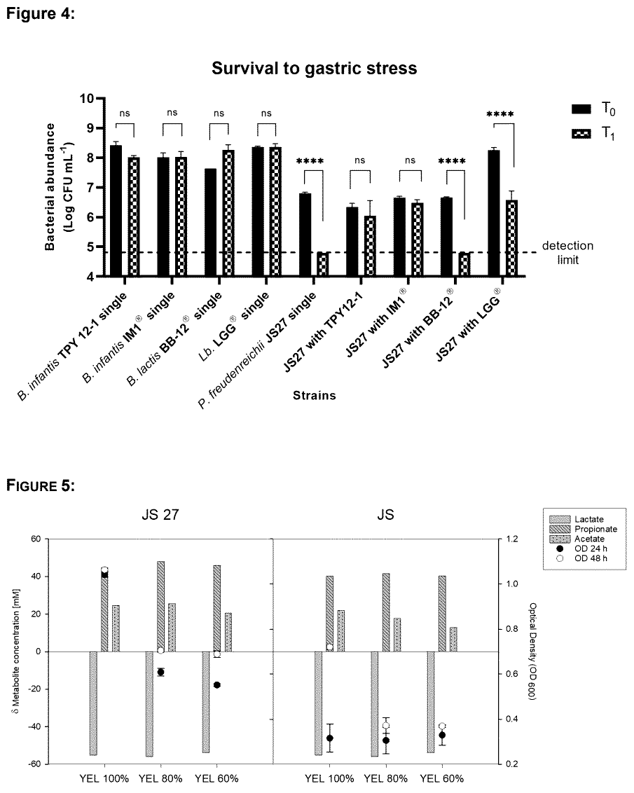

P. freudenreichii JS27 (comprising SEQ ID NO: 7) was selected, before identifying the unexpected synergistic behavior with B. longum subsp. infantis TPY12-1 from a collection of P. freudenreichii strains because it showed faster utilization of lactate and lactose at 24 h (FIG. 3) and higher cell growth in medium supplemented with effluent mimicking the infant colon milieu compared to other strains (FIG. 5). The previous characteristics do not apply to all the P. freudenreichii strains and represent specific properties of P. freudenreichii JS27 to maintain viability and activity in the infant gut.

As shown in FIG. 5, P. freudenreichii JS27 growth was much higher (>two-fold) and metabolite production was increased in culture media containing fermentation effluent (20 and 40%) compared to P. freudenreichii JS DSM 7067.

### Example 5

### Growth and Metabolism of Bifidobacterium Longum in Culture Media Containing Fermentation Effluent

Bifidobacterium longum strains (detailed in Example 1/Table 1) were grown on mWCSP. Glycerol stocks stored at −80° C. were re-activated on liquid media incubated for 48 h at 37° C. and sub-cultured in liquid media before being used as working cultures.

The evaluation of growth of different B. longum strains in presence of prebiotics and filter sterilized fermentation effluent from an in vitro continuous fermentation model mimicking the proximal colon of a two months old infant and inoculated with immobilized fecal microbiota from a two months old bottle-fed healthy baby, corresponding to Fermentation 1 described in Pham et al. (2019), was done in triplicates in 2.2 mL 96-deep-well plates (Milian SA, Vernier/Geneve, Switzerland) covered with Breathe-Easy sealing membranes (Sigma-Aldrich) and incubated in anaerobic jars (Mitsubishi AnaeroPack, Thermo Fisher Diagnostics AG) containing the AnaeroGen system (Oxoid, Thermo Fisher Diagnostics AG) for 24 h at 37° C. Each well contained 1.6 mL of fresh mWCSP medium containing 1.03% galacto-oligosaccharides Vivinal® GOS (Friesland Campina, Netherlands) and 0.08% fructo-oligosaccharides Fibrulose F97 (FOS; COSUCRA, Warcoing, Belgium) with different proportions of fermentation effluent. Water was replaced in media by fermenter effluent in 20% (mWCSP 80%+GOS+FOS) and 40% (mWCSP 60%+GOS+FOS). Wells were inoculated with 16 μL (1% v/v) of each working culture. Cell growth was assessed in triplicates for each strain by measuring culture optical density at 600 nm (OD₆₀₀) at inoculation and at 24 h. Concentrations of substrate, SCFA and intermediate metabolites in supernatant from one sample per strain and per media condition were determined by high-pressure liquid chromatography analysis with refractive index detection (HPLC-RI) after 24 h.

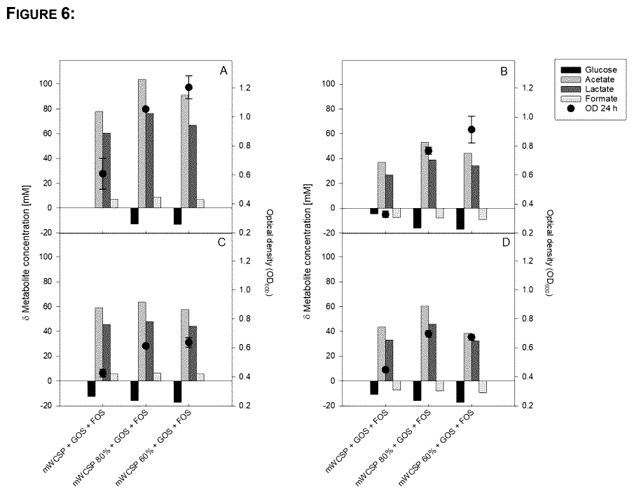

B. longum subsp. infantis TPY12-1 was selected, apart from the unexpected synergistic behavior with P. freudenreichii JS27 from a collection of B. longum strains because cell growth and metabolism after 24 h in medium supplemented with 20 or 40% effluent mimicking the infant colon milieu were highest compared to other B. longum strains (FIG. 6). The previous characteristics do not apply to all B. longum strains and represent specific properties of B. longum subsp. infantis TPY12-1 to maintain viability and activity in the infant gut.

### Example 6

Screening of QPS Propionibacterium strains able to grow at 37° C.

P. freudenreichii JS27 and P. freudenreichii JS DSM 7067 stored in glycerol stocks at −80° C. were reactivated on YEL agar plates and incubated for 5 days in anaerobic jars containing the AnaeroGen system at 30° C. and 37° C. Growth on agar plates incubated at different temperature was visually evaluated.

TABLE 3

| Strain | Growth at 30° C. | Growth at 37° C. |
|---|---|---|
| P. freudenreichii JS27 | +++ | ++ |
| P. freudenreichii JS DSM 7067 | +++ | + |

### REFERENCES

Barr R.G., St James-Roberts I., Keefe M.R., editors. 2001. New evidence on unexplained early infant crying: its origins, nature and management. J Pediatr Gastroenterol Nutr 149-164.
Doo E., Chassard C., Schwab C., Lacroix C. 2017. Effect of dietary nucleosides and yeast extracts on composition and metabolic activity of infant gut microbiota in polyferms colonic fermentation models. FEMS Microbiol Ecol 2017:1-14.
FAO/WHO Joint Working Group Report on Drafting Guidelines for the Evaluation of Probiotics in Food. London, Ontario, Canada, April 30 and May 1, 2002
Fischbach M. A. And Sonnenburg J. L. 2011. Eating for two: how metabolism establishes interspecies interactions in the gut. Cell Host Microbiome 10:336-47.
Furet, Jean-Pierre et al. 2009. Comparative assessment of human and farm animal faecal microbiota using Real-Time quantitative PCR. FEMS Microbiol Ecol 68:351-62.
Gupta S. K. 2002. Is colic a gastrointestinal disorder? Curr Opin Pediatr 14:588-92.

Hervé C., Fondrevez M., Cheron A., et al. 2007. Transcarboxylase mRNA: a marker which evidences P. freudenreichii survival and metabolic activity during its transit in the human gut. Int J Food Microbiol 113:303-14.
Hyman, Paul E. Et al. 2006. Childhood Functional Gastrointestinal Disorders: Neonate/Toddler. Gastroenterology 130:1519-26.
Lehtonen L., Korvenranta H., Eerola E. 1994. Intestinal microflora in colicky and noncolicky infants: bacterial cultures and gas-liquid chromatography. J Pediatr Gastroenterol Nutr 3:310-314.
Macfarlane G.T. and Gibson G.R. 1997. Carbohydrate fermentation, energy transduction and gas metabolism in the human large intestine. In: Mackie, R.I., White, B.A. (Eds.) Gastrointestinal Microbiology, Chapman & Hall Microbiology Series. Springer, Boston, MA.
Mckay L. F., Holbrook W. P., Eastwood M A., 1982. Methane and hydrogen production by human intestinal anaerobic bacteria. Acta Pathol Microbiol Immunol Scand B 3, 257-260.
Meile, L.; Le Blay, G.; Thierry A. 2008. Safety assessment of dairy microorganisms: Propionibacterium and Bifidobacterium. International Journal of Food Microbiology 126:316-320.
Mozzetti, V.; Grattepanche, F.; Berger, B.; Rezzonico, E.; Arigoni, F.; Lacroix, C. 2013. Fast screening of Bifidobacterium longum sublethal stress conditions in a novel two-stage continuous culture strategy. Beneficial Microbes 4; 167-178.
Pham VT, Chassard C, Rifa E, Braegger C, Geirnaert A, Rocha Martin VN, Lacroix C. 2019. Lactate metabolism is strongly modulated by fecal inoculum, pH, and retention time in PolyFermS continuous colonic fermentation models mimicking young infant proximal colon. mSystems 4: e00264-18.
Pham V. T., Lacroix C., Braegger C. P. And Chassard C. 2017. Lactate-Utilizing Community Is Associated with Gut Microbiota Dysbiosis in Colicky Infants. Sci Rep 7:1-13.
Rinttilä, T. et al. 2004. Development of an extensive set of 16S rDNA-targeted primers for quantification of pathogenic and indigenous bacteria in faecal samples by Real-Time PCR. J Appl Microbiol 97:1166-77.
Rocha Martin, V. N., Schwab C., Krych L. Et al. 2019. Cutibacterium avidum is phylogenetically diverse with a subpopulation being adapted to the infant gut.” FEMS Microbiol Ecol 95: fiy215.
Savino F., Cordisco L., Tarasco V., et al. 2009. Molecular identification of coliform bacteria from colicky breastfed infants. Acta Paediatr 98:1582-88.
Savino F., Quarteri A., De Marco. Et al. 2017. Comparison of formula-fed infants with and without colic revealed significant differences in total bacteria, Enterobacteriaceae and faecal ammonia. Acta Paediatr 4:573-578.
Suzuki, A., Mikako I. Hamaguchi T. Et al. 2018. Quantification of hydrogen production by intestinal bacteria that are specifically dysregulated in Parkinson's disease. Plos One 12, e0208313.
Talvik I., Alexander R. C., Talvik T. 2008. Shaken baby syndrome and a baby's cry. Acta Pediatr 97:782-785.
de Weerth C., Fuentes S., Puylaert P. And de Vos W. M. 2013. Intestinal microbiota of infants with colic: development and specific signatures. Pediatrics 131: e55 [symbol]
Zeevenhooven J, Koppen I. J. N, Benninga M. A. 2017. The New Rome IV Criteria for Functional Gastrointestinal Disorders in Infants and Toddlers. Pediatr Gastroenterol Hepatol Nutr 20:1-13.
Zeevenhooven J., Browne P. D., L'Hoir M. P., et al. 2018. Infant colic: mechanisms and management. Nat Rev Gastroenterol Hepatol 15:479-96.

1.-21. (canceled)

22. A bacterial composition comprising viable bacteria of the strain P. freudenreichii JS27 and of the species Bifidobacterium longum subsp. infantis wherein the bacteria of the strain P. freudenreichii JS27 is a bacterium comprising a 16S rDNA sequence as defined by SEQ ID NO: 7 or a sequence having at least 95% sequence identity to SEQ ID NO: 7, wherein the P. freudenreichii JS27 maintains being capable of growing in a colon and wherein post-stress survival is improved by co-culture of the bacterium of the species Bifidobacterium longum subsp. infantis.

23. The composition of claim 22, wherein the bacteria of the strain P. freudenreichii JS27 is a bacterium comprising a sequence as defined by SEQ ID NO: 7.

24. The composition of claim 22, wherein the bacteria of the species B. longum subsp. infantis are of the strain B. longum subsp. infantis TPY12-1.

25. A co-culture of viable bacteria of the strain P. freudenreichii JS27 and of the species Bifidobacterium longum subsp. infantis wherein the bacteria of the strain P. freudenreichii JS27 is a bacterium comprising a sequence as defined by SEQ ID NO: 7 or a sequence having at least 95% sequence identity to SEQ ID NO: 7, wherein the P. freudenreichii JS27 maintains being capable of growing in a colon and wherein post-stress survival is improved by co-culture of the bacterium of the species Bifidobacterium longum subsp. infantis.

26. The co-culture of claim 25, wherein the bacteria of the strain P. freudenreichii JS27 is a bacterium comprising a sequence as defined by SEQ ID NO: 7.

27. The co-culture of claim 26, wherein the bacteria of the species B. longum subsp. infantis are of the strain B. longum subsp. infantis TPY12-1.

28. A method of co-cultivating bacteria of the strain P. freudenreichii JS27 and of the species B. longum subsp. infantis, the method comprising the steps of:
(a) providing a cultivation medium;
(b) inoculating the medium of (a) with bacteria of the strain P. freudenreichii JS27 and of the species B. longum subsp. infantis, preferably of the strain B. longum subsp. infantis TPY12-1;
(c) cultivating the inoculated medium of (b).

29. A method for delivering bacteria of the strain P. freudenreichii JS27 and of the species B. longum subsp. infantis to the gut of a subject, the method comprising administering the composition of claim 22 to said subject.

30. The method of claim 29, comprising oral intake of the composition.

31. The method of claim 29, wherein the subject is an infant.

32. The method of claim 31, wherein the infant is 0 to 3 years of age.

33. A method for ameliorating digestive conditions in the human gut, the method comprising the step of administering the composition of claim 22 to a subject.

34. The method of claim 33, wherein the subject is an infant.

35. The use of claim 34, wherein the infant is of the age of 0 to 3 years

36. A method for treating and/or preventing a digestive disorder in a subject in need thereof, the method comprising the step of administering the composition of claim 22 to said subject.

37. The method of claim 36, wherein the digestive disorder is lactose intolerance, colic, intestinal discomfort, intestinal pain, visceral sensitivity or intestinal cramp.

38. The method of claim 36, wherein the composition is administered orally.

39. The method of claim 36, wherein the subject is an infant.

40. The method of claim 39, wherein the infant is of the age of 0 to 3 years.

41. A food product comprising the composition of claim 22.
